(19) United States
(12) Patent Application Publication
(10) Pub. No.: US 2001/0025166 A1
(43) Pub. Date: Sep. 27, 2001

(54) Abdominal wound dressing with straps and associated elastic strips

(76) Inventor: Jeanette P. Campbell, Florissant, MO (US)

Correspondence Address: Paul M. Denk, 763 South New Ballas Road, St. Louis, MI, 63141, US

(21) Appl. No.: 09/795,526

(22) Filed: Feb. 27, 2001

Related U.S. Application Data
(60) Provisional application No. 60/185,488, filed on Feb. 28, 2000.

(51) Int. Cl. A61F 13/15

(52) U.S. Cl. 604/397; 604/396

(57) ABSTRACT

An abdominal wound dressing incorporating straps, including a pair of straps arranged of substantial size for locating laterally of an abdominal wound incision, having a pressure sensitive adhesive applied, and covered, on its internal surfaces, for direct application to the sides of the abdomen, adjacent the incision, while the upper inner surfaces of the straps have associated therewith one of the loop or pile segments of a Velcro fastener, and cooperating with one or more strips, of an elastic and stretchable type, having the other segment of the Velcro component applied interiorly thereto, adjacent their ends, for removable attachment directly to the previously applied straps, for supporting and holding a wound dressing, and perhaps also an ostomy accessory, in place during abdominal wound healing.

### CROSS REFERENCE TO RELATED APPLICATION

This application is a non-provisional patent application based upon a provisional application filed on Feb. 28, 2000, under Ser. No. 60/185,488, of the same inventor.

### BACKGROUND OF THE INVENTION

Currently, as is well known in medical circles, abdominal surgery, after completion, is normally left to heal from the inside out, leaving a major opened abdominal wound. The wound averages a length of approximately twelve inches, down to the center of the abdomen. The wound is often accompanied with an ostomy. Usually, such wound is approximately two inches to the left of the navel area, and there is normally located therewith the ostomy and its attended paraphernalia. A doctor will normally examine the wound daily, if not more frequently. Nurses examine the wound multi-daily, to assure proper healing, and to detect any incipient infection. Additionally, the wound has a dressing, that also must be changed, daily, if not more frequently. The ostomy bag which catches the fecal material also requires multi-daily attention from the nurses and/or patient.

Upon completion of the surgery, the patient's abdomen is outfitted with a device generally referred to in Medical circles as the Montgomery dressing. This device consists of two Dermicel brand Montgomery Straps, normally sold under Stock No. 5129, and includes eight large safety pins, and four large, wide rubber type bands. The purpose is to stabilize the gauze and dressing covering the long incision. Generally, a Montgomery Strap is approximately seven and a quarter inches by eleven and one-eight inches in size, and consists of a large piece of adhesive tape with a peel-off backing. A single edge of the eleven and one-eight inch side of the dressing has three-inch flap of clear plastic, with perforations along the entire length. These perforations are of a size, and appear to be the result of a hole-punch system, and are spaced vertically apart approximately one inch. A Montgomery dressing or strap is affixed to each side of the patient's abdomen, with the clear plastic having the arranged holes directed towards the incision. With an ostomy, the left clear plastic strap is fitted over and presses on the ostomy. Large safety type pins are anchored to the plastic flaps of the double Montgomery strap, using the perforated holes as the entry/exit positions, which generally form the anchor positions for the two adhered straps. Once the safety pins are in place, the rubber bands are used to span the incision, the wound dressing, the gauze, and the ostomy, to provide means for securing the dressing place, to hold the ostomy in location, during the healing process. A single large rubber band is looped on a single large pin, on one side of the incision, and stretched across the incision to the other side, and looped around the second safety type pin. This is done a total of four times, utilizing the eight safety pins, four on each side, and the four rubber bands to create a support structure for the gauze and its dressing. An alternative to the safety pins and rubber bands is a technique of lacing long gauze strips through the hole to create a support for the gauze and the dressing.

One of the problems associated with the treatment of such an abdominal wound, is that because the number of times that the wound or ostomy requires attention each day, the latter defined gauze type lacing is normally not used in many hospitals. The only current and alternative available dressing that allows multiple examinations, and dressing changes, is the above defined safety pin/rubber band combination, of the Montgomery strap. However, this technique is very time consuming, and labor intensive for the doctors/caregivers, and provokes the patient, during examination and treatment. The numerous rubber bands are easy to remove, but they break, the safety pins become askew, after repeated usage, and within a short time, they may cause tearing to the lateral strap, and frequently during extended usage, may form rust, because of the ambient moisture. The entire technique is unyielding, unsafe, and creates general caregiver/patient dissatisfaction. The rubber bands crush the ostomy bag, making it difficult to service and manage. The ostomy is accessible only by removing all of the rubber bands, and folding back the plastic edge of the left Montgomery strap.

### SUMMARY OF THE INVENTION

This current invention has as its principal object the provision of an abdominal wound dressing and associated strap, that can be used for providing a coverage for an abdominal incision, during its healing process, and yet affords the hospital doctors and nurses the ability to readily remove the dressing, inspect the wound, and immediately place the dressing back into a service condition, with a minimum effort and little or no detraction from the operative attributes of the inventive dressing.

The inventor's development comprehends an alteration to the existing style of Montgomery strap, in that it replaces the perforated plastic edge associated with a Montgomery type strap, with an edge that is formed to furnish an industrial strength type of Velcro, of approximately two inches, more or less, wide, Velcro being the usual type of hook and pile form of fastening means. The pile side of the Velcro fastener may be attached to the lateral strap, with an industrial adhesive, stitching, or any other type of means for adhering one component of the Velcro to such a strap. The patient's body, which underlies the Velcro, is protected by the plastic backing of the formed strap, so that there is no contact of the body by any of the connecting means, such as this style of hook and pile fastener, during usage. The second part of the alteration that enhances the effective usage of this dressing is the provision of elastic strips, approximately one and a half inches wide, more or less, and which are provided, on each end, with the second or hook part of the Velcro fastener, that grabs onto the pile component of the Velcro, as previously described, adhering to the ends of the formed strap. The Velcro fastener, the hook side, on the ends of the elastic strips or straps should be approximately the same length as the pile component of the Velcro previously adhered to the strap of the dressing, and will be about the same width of the underlying pile to provide adequate area for securement to the pile disposed edge of the dressing strap. Because of the stress placed upon the elastic strips, the Velcro, the hook component, will be sewn to or secured by any other means, such as a strong adhesive, to the elastic strip, to assure its retention, even when the strips and the underlying dressing are adhered together, under tension biasing conditions, because of the elasticity of the straps, when the strip is applied to the dressing to assure continued adherence, during prolonged application while the patient convalesces. Three elastic such strips are considered necessary to secure the gauze and dressing in place, although a greater number of such strips may be applied, or even a lesser number, depending upon the width of the strips used. These are required to secure the emplacement of the gauze and the dressing. These elastic strips may be applied straight across the abdomen, or may be applied in a criss cross manner, as necessary, and at the discretion of the doctor or caregiver. Two different lengths of such elastic strips may be used, or may be necessary, to satisfy the patient's individual requirement.

An example of the foregoing, if an ostomy is present, and being applied, the elastic strips identified previously may be as long as approximately eight inches, and will allow the left altered Montgomery type strap or dressing to be affixed onto the patient's body completely laterally and behind the ostomy area, so as to allow freedom for maintenance and service of the ostomy. The lateral strap may be trimmed at an angle to fit the area retaining the pile edge necessary to use the elastic strip, to hold the entire dressing, and the ostomy componentry, in place. The longer elastic strips will fit around, above, or below, as necessary, to allow freedom for the management of the ostomy bag, and to allow for its proper repeated servicing. If there is no ostomy present, or if the patient is small, shorter elastic type strips may be designated, and used, such as approximately five inches, more or less, long, and are available, through this invention, to adequately serve the different patient needs, as required.

Each of these three strips as previously described, such as the eight inch type strip, and the five inch type strip, may be supplied with the lateral straps, as specified above, in an accessory kit, and may come in various sizes, such as long or short, heightwise, as may be necessary for usage by the particular size of the patient involved. Two of the alternative type Montgomery straps, which form the lateral straps of this invention, are used as a set, one for emplacement at each lateral size of the wound, but they are not reusable, and may be disposable, for sanitary reasons. However, if desired, the elastic type strips, formed of the accessory kit, may possibly be laundered and reusable, or simply may be disposable, as required. The inventor suggests and recommends, through experience, that two such accessory kits should be provided for each patient, one required as a backup.

It is, therefore, the principal object of this invention to provide an abdominal wound dressing incorporating readily removable elastic strips that can provide for prompt application, or release, as when an inspection of the wound is required, or ostomy servicing necessary, and which strips can be readily reapplied to complete the dressing during incision healing.

Another object of this invention is to provide an abdominal wound dressing that can be reapplied, for repeat usage.

A further object of this invention is to provide an abdominal dressing that incorporates multisized strips to assure that all types of wound dressings, including accessories such as ostomy, can be readily accommodated by the single dressing of this invention.

Still another object of this invention is to provide an abdominal dressing wherein select components are washable, for repeat usage, and to diminish the potential for producing infection.

Another object of this invention is to provide a particular type of fastenable strip, incorporating the loop and pile type of fastener, that may be stretched by means of its elastic component across a wound dressing, so as to keep the dressing in place, and overlie or underlie the ostomy mechanism, used during incision healing.

These and other objects may become more apparent to those skilled in the art upon reviewing the invention as described herein, and upon undertaking an study of the description of its preferred embodiment, in view of the drawings.

### BRIEF DESCRIPTION OF THE DRAWINGS

In referring to the drawings, FIG. 1 provides a front view of the abdominal wound dressing and its incorporated strap applied in combination with an ostomy, and wound dressing, during the healing of an operative incision;

FIG. 2 provides a front view of the wound dressing and straps of the invention, applied in combination with ostomy;

FIG. 3 is a top exploded view of the dressing of FIG. 2;

FIG. 4 is a front view of a wound dressing of this invention, of the type applied without an ostomy; and

FIG. 5 is an exploded view of the wound dressing of FIG. 4.

In referring to the drawings, and in particular FIG. 1, a patient P is disclosed having an abdominal wound dressing, as at D, applied thereto, overlying the general abdominal area. The dressing, as can be noted, includes a pair of lateral straps, as at 1 and 2, with each of these straps being formed of a heavy linen, gauze, or related type of material. The underside of the straps incorporate a pressure sensitive adhesive, as at 3 and 4, and which adhesive becomes exposed, when their overlying covering sheets 5 and 6 are removed, so as to allow the straps 1 and 2 to be applied directly to the lateral surface of the skin, to the side of the abdomen, and the incision, as can be readily noted in FIG. 1.

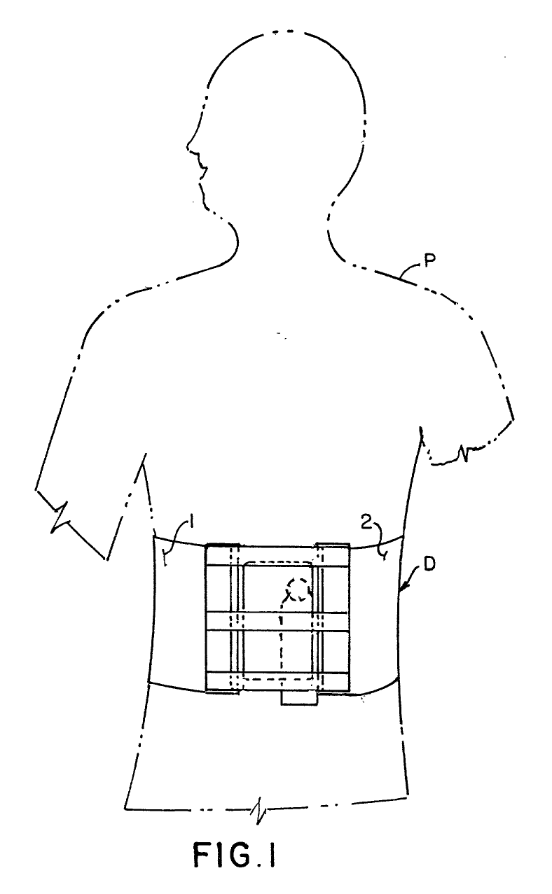

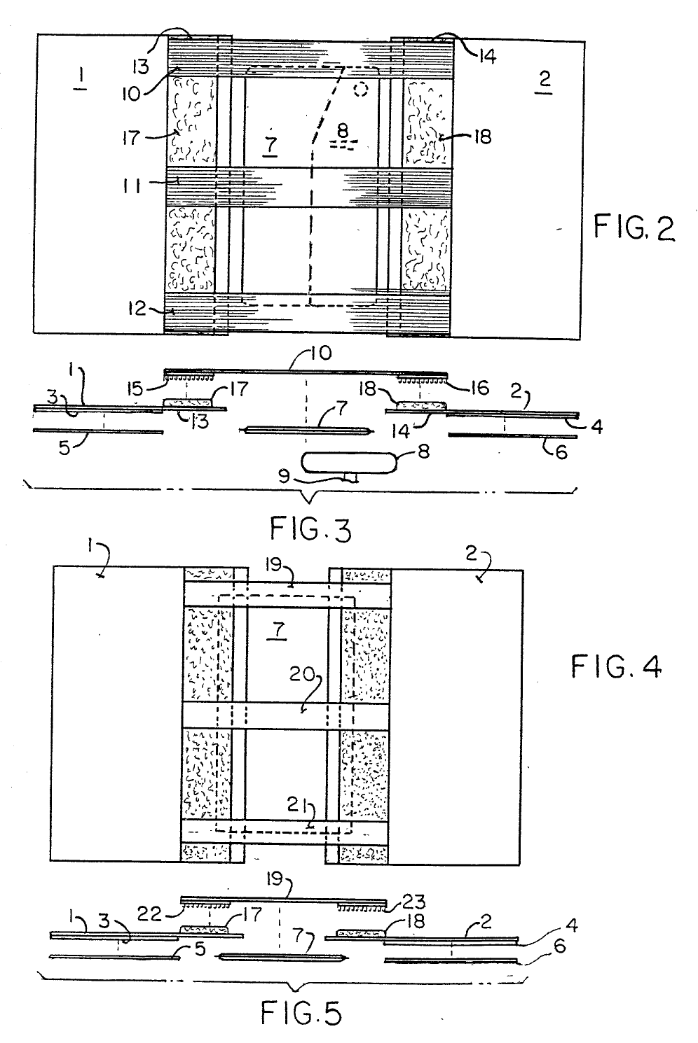

As can also be seen in FIG. 2, a dressing 7 will be applied centrally over the previously formed incision, normally longitudinally and vertically aligned approximately at a central, or slightly offset, of the patient's abdomen. In addition, an ostomy bag 8 with its intake port 9 will be arranged slightly laterally of the incision, to allow for the removal of waste material.

This invention comprehends the usage of elastic style strips, as can be seen at 10 through 12, and which strips are elasticized, as previously summarized, so as to allow for their stretching, between the approximate vertical and inner edges 13 and 14 of the underlying straps 1 and 2, respectively. Velcro, of the hook and type, is applied to the outer but inner surfaces of each of the straps 10 through 12, as can be seen at 15 and 16. These are the hook component of Velcro, applied thereto. In addition, the inner surfaces of the previously defined straps 3 and 4, have rigidly applied thereto, either by stitching, or an industrial type of adhesive, the pile segments 17 and 18 of the previously defined Velcro. In addition, the loop components 15 and 16 of the Velcro may be stitched, or implied by a very strong adhesive, to the approximate ends of their respective elastic strips. Thus, as previously summarized, when it is necessary for the doctor or nurse to inspect to the wound, to determine the success and degree of its healing, the elastic strip 10 can be readily removed, either on one end, or both ends, to allow for removal and replacement of the dressing 7, shift the ostomy 8 laterally, to allow for a quick and prompt inspection of such an incision. And, the strips 10 through 12 can be readily replaced, after providing for their slight expansion, and adherence to the Velcro segments 17 and 18, to promptly reapply a new dressing, any ostomy, in place, during further convalescence.

It should be stated that the type of dressing that is normally applied with the ostomy, is generally wider, in order to add spacing for the ostomy 8 to be located in place, and in this particular instance, the dressing normally has a width of approximately six to seven inches. Hence, the strips 10 through 12 will preferably have a length of approximately eight inches, more or less, when manufactured, and applied.

FIGS. 4 and 5 disclose the type of dressing, as previously explained, but with which an ostomy is not used or present. Hence, the straps 1 and 2 will be configured the same as those as previously described, incorporating the heavy gauze, linen, or even plastic type of segments, laterally disposed of the dressing, and which incorporates the adhesive surfaces 3 and 4, normally covered during shipment and storage by their removable covering sheets 5 and 6, as previously explained. The sheets 5 and 6 may be readily removed, in order to expose the pressure sensitive adhesive 3 and 4, and allow the strips 1 and 2 to be applied laterally of the incision, as noted, and as can be understood. The dressing 7, in this particular instance, since no ostomy is applied, may be somewhat narrower, perhaps in the three to four inch length range, for disposition over the incision, intermediate the inner edges of the straps 1 and 2, as noted. The strips 19 through 21 in this particular instance, will be relative shorter in length, usually in the five inch category, more or less, since greater width is not required when covering just the incision, alone, without the ostomy. In addition, the inner Velcro areas 17 and 18, of the previously defined straps 1 and 2, will be arranged slightly closer together, as a result of the particular emplacement of the straps 1 and 2, since only the incision need be covered by the dressing 7, as explained. The loop segment of the Velcro, as can be noted at 22 and 23, will likewise be arranged proximate the ends of each of the strips 19 through 21, for ready application, or removal, as desired or required, during the treatment of a patient, post operative from the incision.

Once again, the entire dressing, whether it be of the style as shown in FIG. 2, or that explained in FIG. 4, may be disposable, or select components may be cleaned and reusable, particularly when the patient is progressing favorably in his/her her healing, and is being attended to, by a spouse, or other concerned party, as at home.

These are examples of the style of fabrication for abdominal wound dressing and accompanying straps of this invention, and their various alternative embodiments, for use for facilitating, as through prompt application or removal, or replacement, as may be necessary during the convalescence of a patient, following abdominal surgery.

Variations or modifications to the subject matter of this invention may occur to those skilled in the art upon review of the discloses as provided herein. such variations, if within the spirit of this invention, are intended to be encompassed within this disclosure. The description of the preferred embodiments, or as illustrated in the drawings, is set forth just for illustrative purposes.

I claim:

1. An abdominal wound dressing including a pair of lateral straps, provided for arrangement to the side of a surgical wound of the patient, each lateral strap having an outer and interior surface, and also having an inner side edge, at least one elastic strip extending between the pair of lateral straps, a hook and pile fastening means located intermediate the ends of the elastic strips and the inner side edges of the lateral straps, wherein the elastic strips may be temporarily adhered to the lateral straps to locate a wound dressing in place during patient healing, and said elastic strips capable of removal, for replacement of the wound dressing and for treatment of the surgical wound during its healing.

2. The abdominal wound dressing of claim 1 wherein said elastic strips designed for holding a dressing in a position over an ostomy during treatment of a surgical wound of a patient.

3. The abdominal wound dressing of claim 1 wherein there are a pair of elastic strips extending between the lateral straps of the wound dressing.

4. The abdominal wound dressing of claim 1 wherein there are three elastic strips extending between and adhering with the inner side edges of the lateral straps of the wound dressing.

5. The abdominal wound dressing of claim 4 wherein said lateral straps being formed of one of a linen, gauze, or related material, and a pressure sensitive adhesive applied to the interior surface of the lateral straps to provide for their adherence to the skin surface of the patient.

6. The abdominal wound dressing of claim 5 and including covering sheets overlying the pressure sensitive adhesive of the lateral straps during therein their storage and before usage, and wherein said covering sheets capable of removal for exposure of the pressure sensitive adhesive incident to application of the lateral straps to the abdomen of the patient during treatment.

7. The abdominal wound dressing of claim 6 wherein the pile component of the hook and pile connector being adhered to the outer surface at the inner edge of the lateral straps, and the hook component of the hook and pile connector being adhered to the outer interior edges of the elastic strips.

8. The abdominal wound dressing of claim 7 wherein said lateral straps extend a substantial distance to the side of the abdomen to assure its adherence to the abdomen during usage of the wound dressing in treatment of the surgical wound of the patient.
